(19) United States
(12) Patent Application Publication
(10) Pub. No.: US 2019/0167557 A1
(43) Pub. Date: Jun. 6, 2019

(54) COSMETIC COMPOSITION FOR TREATMENT OF KERATINOUS FIBERS

(71) Applicant: Henkel AG & Co. KGaA, Duesseldorf (DE)

(72) Inventors: Sabrina Jasmin Zirwen, Hamburg (DE); Hartmut Manneck, Barnitz (DE)

(73) Assignee: Henkel AG & Co. KGaA, Duesseldorf (DE)

(21) Appl. No.: 16/209,587

(22) Filed: Dec. 4, 2018

Related U.S. Application Data
(60) Provisional application No. 62/624,342, filed on Jan. 31, 2018.

(30) Foreign Application Priority Data
Dec. 4, 2017 (DE) 10 2017 221 773.1

(51) Int. Cl. A61K 8/891; A61K 8/34; A61K 8/37; A61K 8/86; A61K 8/58; A61K 8/81; A61Q 1/10; A61Q 5/06

(52) U.S. Cl. CPC A61K 8/891; A61K 8/342; A61K 8/345; A61K 8/37; A61K 8/86; A61K 8/062; A61K 8/8182; A61Q 1/10; A61Q 5/065; A61K 2800/436; A61K 2800/622; A61K 8/585

(57) ABSTRACT

The present disclosure relates to a cosmetic composition for treatment of keratinous fibers, particularly for the temporary coloring of keratinous fibers, for example as a hair mascara or as a coloring agent for eyebrows. The composition contains water, at least one nonionic oil-in-water emulsifier, at least one hydrophobized pigment, polyphenylmethylsiloxane and at least one linear 1-alkanol with from about 14 to about 30 carbon atoms which is solid at 30° C., wherein the total amount of soap, relative to the weight of the cosmetic composition, is less than about 0.5 wt. % and particularly about 0 wt. %, and wherein the total amount of polysiloxanes, with the exception of polyphenylmethylsiloxane, relative to the weight of the cosmetic composition, is from about 0 to about 1 wt. % and particularly about 0 wt. %.

### CROSS-REFERENCE TO RELATED APPLICATION

This application claims priority to U.S. Provisional Patent Application No. 62/624,342, filed Jan. 31, 2018, and to German Patent Application No. 10 2017 221 773.1, filed Dec. 4, 2017, which are all hereby incorporated in their entirety by reference.

### TECHNICAL FIELD

The present disclosure relates to a cosmetic composition and a method for treatment of keratinous fibers, uses of the cosmetic composition and a hair mascara or eyebrow coloring agent containing the composition.

### BACKGROUND

The dyeing of human body hair is an important field of decorative cosmetics. In the process, there is a differentition between permanent dyeing and temporary dyeing. Permanent dyeing should not be removable from the hair even after intensive washing and have high fastness with respect to environmental influences, such as UV light, sweat and moisture. Corresponding coloring agents are normally formulated on the basis of oxidation dye precursors. Temporary colorings are normally formulated on the basis of partially-oxidizing dyes or pigments that are insoluble in water and do not normally contain any oxidation dye precursors. Such colorings can be practically removed from the hair again with multiple intensive washings.

In the context of modern fashion trends, however, there is an increasing requirement for coloring effects in the hair, which can also be achieved easily without involvement of a hairdresser and can also be removed again easily, i.e. with a single normal washing of the hair. In particular, the corresponding coloring effects should also be limited to individual hair strands or hair sections. In addition, it is highly desirable that the color effects can also be provided on the already completely styled hairstyle.

A product group which could basically fulfill these requirements is mascaras. In the process, however, a major problem is the drying times and the associated consistency of the product while it remains on the hair. If the drying time is too short, the product dries during the application on the hair so quickly that corrections of the application, which also includes an even application of the product on a larger surface area of the hair, is hardly possible. Complete drying of the product does not take place until during the dwell time on the hair. This can also result in adhesive bonding of the hair and crumbling of the mascara mass. In addition, a brief freshening or brushing of the hairstyle is hardly possible with application of the product on the hair head. Excessive drying times, by contrast, lead to abrasion of the mascara or a feeling of “dampness” not desired by the user.

### BRIEF SUMMARY

Cosmetic compositions and methods for treatment of keratinous fibers are provided herein. In an embodiment, a cosmetic composition includes water, at least one nonionic oil-in-water emulsifier, at least one hydrophobized pigment, polyphenylmethylsiloxane, and at least one linear 1-alkanol with from about 14 to about 30 carbon atoms which is solid at 30° C. and a pressure of 1013 mbar. Relative to the weight of the cosmetic composition, the at least one nonionic oil-in-water emulsifier is present in a total amount of from about 2 to about 10 wt. %, the at least one hydrophobized pigment is present in a total amount of from about 3 to about 15 wt. %, an amount of soap is less than 0.5 wt. %, and a total amount of polysiloxanes, with the exception of polyphenylmethylsiloxane, is from about 0 to about 1 wt. %.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present application addresses the problem of providing an agent for temporary dyeing of keratinous fibers, particularly hair on the head and particularly the hairline, as well as for temporary dyeing of eyebrows, such that premature and complete drying are prevented to the extent that the keratinous fibers can still be styled and combed after a wearing time without the remaining ingredients or components detaching from the application area after the cosmetic composition has dried on the keratinous fibers.

It has now been found that a specific combination of ingredients can provide a cosmetic composition for treatment of keratinous fibers which fulfills the requirements for a sensation of dry hair can be prepared and, in the process, prevents premature and complete drying of the hair in this respect, so that the keratinous fibers can still be styled and combed after a certain wearing time of the ingredients of the cosmetic composition remaining on the hair without the constituents or components remaining on keratinous after the drying of the cosmetic composition detaching from the application area, for example in the form of dandruff or flakes.

The subject of the present disclosure, therefore, is a cosmetic composition for treatment of keratinous fibers containing water,
at least one nonionic oil-in-water emulsifier in a total amount, relative to the weight of the cosmetic composition, of from about 2 to about 10 wt. %, preferably from about 3 to about 8 wt. %, more preferably from about 4 to about 6 wt. %, particularly from about 4.5 to about 5.3 wt. %,
at least one hydrophobized pigment in a total amount, relative to the weight of the cosmetic composition, of from about 3 to about 15 wt. %, preferably from about 4 to about 12 wt. %, more preferably from about 6 to about 10 wt. % and even more preferably from about 7 to about 9 wt. %, polyphenylmethylsiloxane and
   at least one linear 1-alkanol with from about 14 to about 30 carbon atoms which is solid at 30° C. and a pressure of 1013 mbar,
wherein the amount of soap, relative to the weight of the cosmetic composition, is less than about 0.5 wt. % and particularly about 0 wt. %, and
wherein the total amount of polysiloxanes, with the exception of polyphenylmethylsiloxane, relative to the weight of the cosmetic composition, is from about 0 to about 1 wt. % and particularly about 0 wt. %.

The first component of the cosmetic composition as contemplated herein is water. Water is the base of the cosmetic composition and enables, depending on the amount added, adjustment of the fluidity of the cosmetic composition so that an initial application already achieves an adequate application of mass on the keratinous fibers without the cosmetic composition running off of the application area. Water is adequately volatile under normal operating temperatures so that the cosmetic composition obtains a good drying rate as a result.

For an especially consistent application, a high proportion of water in the cosmetic composition is advantageous, whereas very high proportions of water promote an undesired running off of the cosmetic composition from the application area. Therefore, the total amount of water, relative to the weight of the cosmetic composition, is from about 40 to about 80 wt. %, preferably from about 50 to about 75 wt. %, more preferably from about 60 to about 70 wt. % and especially from about 63 to about 68 wt. %. If the total amount of water is in a range of from about 63 to about 68 wt. %, cosmetic compositions having especially good application and drying characteristics can be achieved.

However, in order to force the drying of the cosmetic composition and to enable a certain styling and/or even enable combability of the keratinous fibers after application of the cosmetic composition on the keratinous fibers, polyphenylmethylsiloxane is contained in the cosmetic composition, as contemplated herein, as an additional essential component.

Polyphenylmethylsiloxane is a siloxane polymer that can be represented by the following chemical formula, wherein x is preferably from about 1 to about 3:

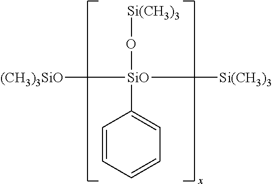

In comparison with polydimethylsiloxanes normally used in mascara or hair mascara, the polyphenylmethylsiloxane is exemplified by an oilier character at a temperature of 25° C. and a pressure of 1013 mbar, which does not have any burdening effect with application on keratinous fibers. This is especially advantageous for the shapeability and/or combability of the keratinous fibers after application of the cosmetic composition as contemplated herein. Small amounts of the polyphenylmethylsiloxane also improve the drying behavior of the cosmetic composition such a rigid film does not remain on the keratinous hair on application and even after drying of the cosmetic composition, rather that the cosmetic composition and/or the residual parts there of leave a type of flexible film behind after drying, which feels dry, but does not result in crumbling or detachment of the cosmetic composition of keratinous fibers.

To achieve the aforementioned advantageous effects, it is preferable that the total amount of at least one polyphenylmethylsiloxane, relative to the weight of the cosmetic composition, is from about 0.05 to about 10 wt. %, preferably from about 0.5 to about 7 wt. %, more preferably from about 0.7 to about 3 wt. %, even more preferably from about 0.8 to about 2 wt. % and particularly from about 1 to about 1.5 wt. %. While a total amount of polyphenylmethylsiloxane of less than about 0.05 wt. % enables a shapeability of the keratinous fibers after application of the cosmetic composition, under certain circumstances the combability of the keratinous fibers cannot always be achieved to a high degree after the drying of the cosmetic composition. In other words, the flexibility of the formed film can be reduced after the drying of the cosmetic composition. A total amount of polyphenylmethylsiloxane of at least about 0.05 wt. %, preferably at least about 0.5 wt. %, more preferably at least about 0.7 wt. %, particularly preferably at least about 0.8 wt. % and most preferably at least about 1 wt. % is especially well-suited to enable good combability and good shapeability of the keratinous fibers to which the cosmetic composition is applied. In the process, the great the share of polyphenylmethylsiloxane is, the more extensive and more flexible the generated film is after the drying of the cosmetic composition on the keratinous fibers. However, in order to also factor in the requirement of the user for a particularly dry feeling of the hair, the at least one polyphenylmethylsiloxane is preferable used in a maximum total amount of about 10 wt. %, more preferably about 7 wt. %, even more preferably about 5 wt. %, especially preferably at least about 2 wt. % and particularly preferably about 1.5 wt. %, relative to the weight of the cosmetic composition. The amounts specified here are also especially well-suited, in combination with the at least on hydrophobized pigment contained in the cosmetic composition, form a consistent color film on the application area and, in the process, also lend a certain shine to the keratinous fibers.

In order not to impair the film formation, as contemplated herein it is also preferred that the total amount of polysiloxanes, with the exception of polyphenylmethylsiloxane, relative to the weight of the cosmetic composition, is from about 0 to about 1 wt. % and particularly about 0 wt. %. This means that only the polyphenylmethylsiloxane as a polysiloxane compound is a component of the cosmetic composition, with the exception of unavoidable technical impurities, such that, in other words, other polysiloxanes, such as cyclical polydimethylsiloxanes, particularly hexamethyl cyclotrisiloxane, octamethylcyclotetrasiloxane, decamethylcyclopentasiloxane or dodecamethylcyclohexsiloxane, or linear polysiloxanes, such as, in particular, dimethicones, hexamethyldisiloxane or octamethyltrisiloxane are not added to the cosmetic composition. Polysiloxane compounds, such as PEG dimethicones, are not used in the cosmetic composition as contemplated herein. According to the present disclosure, polysiloxanes which should not be used according to the present disclosure should be understood as siloxanes with two or three repeating units containing silicon atoms.

Polysiloxanes, excluding polyphenylmethylsiloxane, are, as contemplated herein, not only unnecessary, but explicitly undesired if they would negatively influence the drying behavior of the cosmetic composition. This applies particularly for polysiloxanes which have a certain fluidity at temperatures above 25° C. and a pressure of 1013 mbar, such as the aforementioned hexamethylcyclotrisiloxanes, octamethylcyclotetrasiloxanes, decamethylcyclopentasiloxane, dodecamethylcyclohexasiloxanes, hexamethyldisiloxanes and octamethyltrisiloxanes. These polysiloxane compounds would accelerate the drying of the cosmetic composition so that the homogeneous application on the keratinous fibers is diminished and they would not remain on the keratinous fibers after application, so that the flexibility of the film formed by the dried cosmetic composition would also be diminished.

By contrast, nonvolatile polysiloxane compounds remain on the keratinous fibers after application and thereby burden the hair. The user has a sensation of moisture of the keratinous fibers even after a long drying time and the combability of the keratinous fibers is reduced. Without being tied to the theory, it is assumed here, in comparison to the polyphenylmethylsiloxane used as contemplated herein, conventionally used non-volatile polysiloxane compounds have lower sliding properties, that is to say have reduced slip, which has a disadvantageous effect on the combing or styling of the keratinous fibers.

For stabilization of the cosmetic compound, the cosmetic compound according to the convention contains at least one nonionic oil-in-water emulsifier (O/W emulsifier) as an additional essential component in a total amount, relative to the weight of the cosmetic composition, of from about 2 to about 10 wt. %, preferably from about 3 to about 8 wt. %, more preferably from about 4 to about 6 wt. %, particularly from about 4.5 to about 5.3 wt. %.

According to a preferred embodiment of the present disclosure, the at least one nonionic oil-in-water emulsifier is selected from ethoxylated C₈-C₂₄ alkanols with from about 10 to from about 100 mol of ethylene oxide (EO) per mole, preferably from about 20 to from about 30 mole EO per mole, ethoxylated C₈-C₂₄ carboxylic acids with from about 10 to about 100 mol of ethylene oxide per mol, preferably from about 20 to about 30 mol EO per mol, with from about 20 to about 100 mol of ethylene oxide per mol of ethoxylated sorbitan monoesters of linear saturated and unsaturated C₁₂ to C₃₀ carboxylic acids which can be hydroxylated, alkyl mono- and oligoglycosides with from about 8 to about 22 carbon atoms in the alkyl radical, which can be ethoxylated, ethoxylated sterines, partial esters of polyglycerols with n=from about 2 to about 10 glycerol units and with from about 1 to about 4 saturated or unsaturated, linear or branched optionally hydroxylated C₈ to C₃₀ fatty acid radicals. In this connection, a nonionic oil-in water emulsifier can be used alone or a mixture of two or multiple nonionic oil-in-water emulsifiers can be used. The aforementioned nonionic oil-in-water emulsifiers have been found to be highly efficiently in stabilizing the cosmetic composition and improving the color uptake on keratinous fibers. Among these, particular preference is given to oil-in-water emulsifiers selected from those having from about 10 to about 100 mol of ethylene oxide per mol, preferably from about 20 to about 30 mol of ethylene oxide per mol of ethoxylated fatty alcohols, particularly from those having from about 10 to about 100 mol of ethylene oxide per mol, preferably from about 20 to about 30 mol of ethylene oxide per mole of ethoxylated cetyl alcohol, with from about 10 to about 100 mol of ethylene oxide per mol, preferably from about 20 to about 30 mol of ethylene oxide per mol of ethoxylated stearyl alcohol and mixtures thereof. Among these, particular preference is given to those having about 20 mol of ethylene oxide per mol of ethoxylated cetyl alcohol (INCI name: Ceteth-20), with about 20 mol of ethylene oxide per mole ethoxylated stearyl alcohol (INCI name: Steareth-20) and mixtures thereof (INCI name: Ceteareth-20).

Surprisingly, it has been found that soaps as O/W emulsifiers do not provide the desired result, specifically a cosmetic composition that is stable during storage.

According to the present disclosure, soaps are understood to mean a fatty acid saponified by employing a base, particularly sodium or potassium salts of fatty acids, wherein fatty acids are monocarboxylic acids with at least about 3 carbon atoms and, optionally, one or multiple hydroxyl groups.

It has been found that soaps do not form adequately storage-stable compositions in the composition as contemplated herein, rather, that they diminish the shapeability and combability of the keratinous fibers after application of the cosmetic composition. The total amount of soaps, relative to the weight of the cosmetic composition, is less than about 0.5 wt. % and particularly about 0 wt. %. It is particularly preferred that no soap is added to the cosmetic composition, in which case a very high stability of the cosmetic composition with good application characteristics can be achieve.

The cosmetic composition as contemplated herein contains, as an additional essential component, at least one hydrophobized pigment, i.e. one hydrophobized pigment by itself or a mixture of two or more hydrophobized pigments.

A hydrophobized in the context of the present disclosure is understood to mean a pigment with a pigment core and a hydrophobically modified pigment surface. This means, in particular, that the pigment surface is treated with a hydrophobic agent so that the normally at least partially present hydrophilic character of commonly used pigments is shifted significantly towards a lipophilic character. As contemplated herein, preference is given to hydrophobic agents selected from organomonosilanes, such as triethoxycaprylylsilane and perfluorooctyltriethoxysilane, and isopropyltitanium triisostearate, preferably of triethoxy-n-octylsilane, which carries the INCI name triethoxycaprylylsilane. Additional suitable hydrophobized pigments are described, for example, in EP 257423A2.

Preferred pigment cores of the at least one hydrophobized pigment are color, effect and/or pearlescent pigments, which can be of organic and/or inorganic origin.

Due to their excellent light, weather and/or temperature fastness, inorganic pigments are particularly preferred as a pigment core as contemplated herein.

The preferred particle size of the—preferred inorganic—hydrophobized pigments is from about 0.1 μm to about 1 mm, preferably from about 0.5 μm to about 100 μm, more preferably from about 1 μm to about 50 μm and particularly preferably from about 5 μm to about 30 μm.

Preferred pigment cores are selected from inorganic pigments which can be of natural origin. Inorganic color pigments of natural origin can be produced, for example, from chalk, ocher, umber, green earth, fired terra di siena or graphite.

Furthermore, white pigments can be used as inorganic pigment cores, including titanium dioxide or zinc dioxide, black pigments, such as iron oxide black, chromatic pigments, such as ultramarine or iron oxide red as well as fluorescent or phosphorescent pigments, wherein preferably at least one pigment is a colored non-white pigment. Metal oxides, metal hydroxides and metal oxide hydrates are particularly well-suited as mixed phase pigments, sulfur-containing silicates, metal sulphides, complex metal cyanides, metal sulphates, metal chromates and/or metal molybdates. Particularly preferred pigment cores are titanium dioxide (CI 77891), black iron oxide (CI 77499), yellow iron oxide (CI 77492), red and brown iron oxide (CI 77491), manganese violet (CI 77742), ultramarine pigments (sodium aluminum sulphosilicates) in particular ultramarine blue (CI 77007, pigment blue 29), ultramarine violet (pigment violet 15), and ultramarine green (pigment green 24), as well as chromium oxide hydrate (CI77289) and/or iron blue (ferric ferrocyanide, CI77510), wherein brown iron oxide (CI 77491) and black iron oxide (CI 77499) and the ultramarine are particularly preferred and thus serve as preferred pigment cores for the hydrophobized pigments used as contemplated herein.

Among suitable effect pigments as a pigment core, particular preference is given to metal effect pigments, such as bronze pigments.

Additional pigments preferably used as pigment cores are pearlescent pigments. They are normally based on mica and/or glimmer and can be coated with one or multiple of the aforementioned metal oxides or a metal oxychloride, such bismuth oxychloride.

As contemplated herein, preference is given to pearlescent pigments based on natural glimmer and on natural glimmer/metal oxide.

Glimmer is included among the coating silicates. The most important representatives of such silicates are muscovite, phlogopite, pargonite, biotite, lepidolite and margarite. To produce the pearlescent pigments in conjunction with metal oxides, the glimmer, primarily muscovite or phlogopite, is coated with a metal oxide.

As an alternative to natural glimmer, synthetic mica coated with one or multiple metal oxides can be optionally used as a pearlescent pigment. Such suitable pearlescent pigments based on natural micas are described in WO 2005/065632, which is expressly incorporated by reference.

Particularly preferred pearlescent pigments are based on natural or synthetic mica (glimmer) and coated with one or multiple of the aforementioned metal oxides.

The color of the respective pigment can be varied by varying the coating thickness of the metal oxide(s).

Cosmetic compositions preferred as contemplated herein are exemplified in that the at least one hydrophobized pigment contains a pigment core, which is selected from the group of metal oxides, metal oxychlorides, metal hydroxides and metal oxide hydrates, mixed phase pigments, sulfur-containing silicates, metal sulphides, complex metal cyanides, metal sulphates, metal phosphates, metal chromates, metal molybdates, fluorescent pigments, phosphorescent pigments, metallic effect pigments, in particular bronze pigments, phyllosilicates, in particular micas, in particular micas of the muscovite sub-group, the phlogopite sub-group, the pargonite sub-group, the biotics sub-group, the margarite sub-group and the lepidolite series, pearlescent pigments based on natural or synthetic mica, which are coated with one or more metal oxide or a metal oxychloride, such as bismuth oxychloride, chalk, ocher, umber, green earth, fired terra di siena and graphite, and mixtures thereof, preferably selected from iron oxide black (CI 77499), iron oxide red (CI 77491), iron oxide brown (CI 77491), yellow iron oxide (CI 77492), ultramarine, in particular ultramarine blue (CI 77007, pigment blue 29), ultramarine violet (pigment violet 15), and ultramarine green (pigment green 24), as well as chromium oxide hydrate (CI 77289), iron blue (ferric ferrocyanide, CI 77510), titanium dioxide (CI 77891), manganese violet (CI 77742), and zinc oxide, and mixtures thereof, preferably selected from iron oxide black (CI 77499), iron oxide red (CI 77491), iron oxide brown (CI 77491), iron oxide yellow (CI 77492), ultramarine, in particular ultramarine blue (CI 77007, pigment blue 29), ultramarine violet (pigment violet 15), and ultramarine green (pigment green 24) and mixtures thereof.

Due to the hydrophobic character of the pigment used as contemplated herein, in other words, the predominantly lipophilic character of the hydrophobized pigment, these pigments have a certain affinity to polyphenylmethylsiloxane. This is advantageous, because the improved applicability of the cosmetic composition on the keratinous fibers also promotes homogeneous coloring of the keratinous fibers. The color coverage of the keratinous fibers becomes more consistent with the hydrophobated pigments, so a good, homogeneous application is achieved. Use of pigments modified with triethoxycaprylylsilane, in particular, has demonstrated an especially pronounced effect of consistent color release on keratinous fibers.

The total amount of hydrophobized pigments, wherein the portion of hydrophobic agent is included in this total amount, is from about 3 to about 15 wt. %, preferably from about 4 to about 12 wt. %, more preferably from about 6 to about 10 wt.5 and most preferably from about 7 to about 9 wt. %, relative to the weight of the cosmetic composition as contemplated herein, and can be adjusted in the scope of the specified amounts according to the desired coloring characteristic and in respect to the stability of the cosmetic composition, wherein a very good color release of the cosmetic composition and a high stability of the composition can be achieved with a total amount of from about 7 to about 9 wt. %.

As contemplated herein, for good color uptake, a good coloring capability and good durability on the keratinous fibers, preference is given to hydrophobized pigments having a weight ratio of pigment core to hydrophobic coating material in the range of from about 40 to about 1 to about 18 to about 1, preferably from about 35 to about 1 to about 20 to about 1, more preferably from about 30 to about 1 to about 24 to about 1.

Particularly preferred cosmetic compositions contain at least one pigment hydrophobized with triethoxycaprylylsilane in a total amount, relative to the weight of the cosmetic composition as contemplated herein, of from about 3 to about 15 wt. %, preferably from about 4 to about 12 wt. %, more preferably from about 6 to about 10 wt. % and even more preferably from about 7 to about 9 wt. %, wherein these specified amounts also comprise the portion of hydrophobic agent and wherein the pigment(s) has(have) a weight ratio of pigment core to hydrophobic coating material in the range of from about 40 to about 1 to about 18 to about 1, more preferably from about 35 to about 1 to about 20 to about 1, even more preferably from about 30 to about 1 to about 24 to about 1.

Furthermore, it is essential, as contemplated herein, that the cosmetic composition contains at least one linear 1-alkanol with from about 14 to about 30 carbon atoms which is solid at 30° C. and a pressure of 1013 mbar, As a result, the consistency of the cosmetic composition as contemplated herein can be increased, wherein the nurturing character of the cosmetic composition is improved without reducing the pleasant and smooth application. Preferred linear 1-alkanols are selected from myristyl alcohol, cetyl alcohol, stearyl alcohol, arachyl alcohol, behenyl alcohol, erucyl alcohol and brassidyl alcohol, and mixtures thereof. Particularly preferred linear 1-alkanols are selected from myristyl alcohol, cetyl alcohol, stearyl alcohol, arachyl alcohol, behenyl alcohol, erucyl alcohol and brassidyl alcohol, and mixtures thereof. The linear 1-alkanols with from about 14 to about 30 carbon atoms preferred as contemplated herein improve the stability of the cosmetic composition and prevent, in particular, separation of the hydrophobic components of the compound.

The cosmetic composition as contemplated herein is exemplified by the combination of its essential components with a moderate drying time, i.e. a drying rate that is not excessively high, which enables a precise, consistent application of the cosmetic composition without leaving a moist film on the keratinous fibers after drying completely. Moreover, the cosmetic composition also enables a shapeability and combability of the keratinous fibers after drying on the keratinous fibers. An additional advantageous effect is the omission of volatile cyclic polydimethylsiloxanes, such as hexamethylcyclotrisiloxane, octamethylcyclotetrasiloxane, decamethylcyclopentasiloxane and dodecamethylcyclohexsiloxane, which improves the biodegradability of the cosmetic composition and thus increases market acceptance. The cosmetic composition as contemplated herein is suitable for temporary coloring of keratinous fibers.

Among the aforementioned aspects, it is preferred that the total amount of the at least one linear 1-alkanol with from about 14 to about 30 carbon atoms, which is solid at 30° C. and a pressure of 1013 mbar, is contained in a range of from about 1 to about 20 wt. %, preferably from about 3 to about 15 wt. %, more preferably from about 5 to about 12 wt. % and most preferably from about 8 to about 10 wt. %, relative to the weight of the cosmetic composition.

To improve the film-forming characteristics of the cosmetic composition on keratinous fibers, which also increases the shapeability of the keratinous fibers, the cosmetic composition preferably contains at least one vinyl polymer, which is preferably selected from the group of: polyvinyl alcohol, polyvinyl acetate, polyvinyl pyrrolidone and mixtures and copolymers thereof. Particular preference is given to at least one vinylpyrrolidone/vinylacetate copolymer, wherein the weight ratio of vinylpyrrolidone to vinylacetate is preferably from about 70 to about 30 to about 50 to about 50 and ideally from about 60 to about 40. The relative high portion of vinylacetate is particularly well-suited to achieve a certain water-repellent effect after application of the cosmetic composition on the keratinous fibers. The preferred vinyl polymers are very well-suited to for application on keratinous fibers, at least partially surrounded them to improve the shapeability and also the combability of the keratin fibers even after the cosmetic composition has dried on the keratin fibers.

Among these aspects, the total amount of vinyl polymer and particularly one or multiple vinylpyrrolidone/vinylacetate-copolymers is preferably from about 0.1 to about 10 wt. %, relative to the weight of the cosmetic composition. It is also preferred that the total amount of vinyl polymer is from about 0.2 to about 6 wt. %, more preferably from about 0.25 to about 2 wt. % and even more preferably from about 0.3 to about 1 wt. %, relative to the weight of the cosmetic composition. Although the film-forming characteristic of vinyl polymers increases as the total amount of polymers increases, especially high amounts of, for example, more than about 6.0 wt. % and particularly more than about 10 wt. % can result in an adhesive bonding of the keratinous fibers, in which case the combability is reduced.

For further optimization of the coloring characteristics of the cosmetic composition, an additional preferred embodiment also contains at least one organic solvent. The organic solvent is selected from glycerol, 1,2-propanediol and 1,3-butanediol and mixtures thereof. This is due to the fact that these solvents are dissolved at a temperature of 25° C. and non-volatile at a pressure of 1013 mbar and are thus retained even after the cosmetic composition has dried, so that the flexibility of the keratinous fibers to which the cosmetic composition was applied can be increased. In particular, the use of glycerol, 1,2-propanediol and 1,3-butanediol and mixtures thereof in the presence of one or multiple vinyl polymers additionally advantageous, because glycerol and 1,2-propanediol and 1,3-butanediol can serve as softeners for the vinyl polymer, so that they can form an especially flexible and thus shapeable film.

The total amount of glycerol, 1,2-propanediol and 1,3-butanediol, relative to the weight of the cosmetic composition is preferably from about 1 to about 15 wt. %, more preferably from about 2 to about 10 wt. %, even more preferably from about 3 to about 8 wt. % and particularly from about 3.5 to about 6 wt. %. The total amounts specified here are on the high side so that the volatility of the cosmetic composition can be influenced advantageously, but not so thigh that a drying of the cosmetic composition after application on the keratinous fibers is prevented to the extent that the keratinous fibers retain a certain moist feeling.

It is additionally preferred that the cosmetic composition contains at least one polyethyleneglycol which is solid at 25° C. and a pressure of 1013 mbar, particularly a polyethylene glycol having from about 25 to about 100 ethylene oxide units, preferably from about 27 to about 50 ethylene oxide units, more preferably from about 28 to about 40 ethylene oxide units and most preferably from about 30 to about 34 ethylene oxide units, wherein the total amount of solid polyethylene glycol, relative to the weight of the cosmetic composition, is from about 0.5 to about 10 wt. %, preferably from about 0.8 to about 5 wt. %, more preferably from about 1.2 to about 3 wt. % and most preferably from about 1.4 to about 1.6 wt. %. As a result, the homogeneity of the cosmetic composition is improved and a dry feeling of the composition is prevented.

An additional advantageous embodiment is exemplified in that the cosmetic composition also contains at least one ester which is formed from at least of at least one linear or branched, saturated or unsaturated monocarboxylic acid with from about 3 to about 30 carbon atoms, which can optionally contain one or multiple hydroxyl groups and at least one linear or branched, saturated or unsaturated alcohol with from about 1 to about 30 carbon atoms and which is solid at 35° C. and a pressure of 1013 mbar. Such esters are advantageous in regard to the application behavior. They give the cosmetic composition a certain softness and optimize the coloring characteristics of the compositions as contemplated herein. In consideration of these aspects, particularly suitable esters are selected from cetyl palmitate, stearyl laurate, cetearyl stearate, and myristyl myristate and mixtures thereof, with particularly preference being given to cetyl palmitate. Cetyl palmitate has been found to be additionally advantageous in combination with the polyphenylmethylsiloxane, because both components support film formation on the keratinous fibers. In an additional preferred embodiment, the cosmetic composition contains the at least one ester, which is formed from at least of at least one linear or branched, saturated or unsaturated monocarboxylic acid with from about 3 to about 30 carbon atoms, which can optionally contain one or multiple hydroxyl groups and at least one linear or branched, saturated or unsaturated alcohol with from about 1 to about 30 carbon atoms and which is solid at 35° C. and a pressure of 1013 mbar, in a total amount of from about 0.1 to about 6 wt. %, preferably from about 0.5 to about 5 wt. %, more preferably from about 1 to about 4 wt. %, even more preferably from about 2 to about 3 wt. %, relative to the weight of the cosmetic composition. It is particularly preferred that at least one ester selected from cetyl palmitate, stearyl laurate, cetearyl stearate, and myristyl myristate and mixtures thereof is contained in a total amount of from about 0.1 to about 6 wt. %, preferably from about 0.5 to about 5 wt. %, more preferably from about 1 to about 4 wt. %, even more preferably from about 2 to about 3 wt. %, relative to the weight of the cosmetic composition. It is especially preferred that cetyl palmitate is contained in a total amount of from about 0.1 to about 6 wt. %, preferably from about 0.5 to about 5 wt. %, more preferably from about 1 to 4 wt. %, even more preferably from about 2 to about 3 wt. %, relative to the weight of the cosmetic composition.

The cosmetic composition can contain additional components, e.g. for nuancing, such as small amounts of partially-oxidizing dyes which wash out easily. Furthermore, the cosmetic composition as contemplated herein can contain all active, additional auxiliary ingredients. Such ingredients include
cationic polymers, such as quaternized cellulose ethers, dimethyldiallylammonium chloride polymers, acrylamide-dimethyldiallylammonium chloride copolymers, dimethylaminoethyl methacrylate-vinylpyrrolidone copolymers quaternized with diethyl sulfate, vinylpyrrolidone-imidazolinium methochloride copolymers and quaternized polyvinyl alcohol,
zwitterionic and amphoteric polymers, such as acrylamidopropyl trimethyl ammonium chloride/acrylate copolymers and octylacrylamide/methyl methacrylate/tert.-butylaminoethyl methacrylate/2-hydroxypropyl methacrylate,
phospholipids, for example soya lecithin, egg lecithin and kephalins,
protein hydrolysates, in particular elastin, collagen, keratin, milk-, soya protein and wheat protein hydrolysates, condensation products thereof with fatty acids and quaternized protein hydrolysates,
substances for adjustment of the pH value of the compositions as contemplated herein and compositions preferred as contemplated herein to a range of from about 4.5 to about 8.0, preferably from about 5.0 to about 7.6, more preferably from about 5.2 to about 7.0, measured at 20° C.,
fiber-nurturing ingredients such as panthenol, pantothenic acid, allantoin, pyrrolidone carboxylic acid and salts thereof, and vitamins and plant extracts,
preservatives and
antioxidants.

A further aspect of the present disclosure relates to a hair mascara or an eyebrow coloring agent which contains the cosmetic composition described above. The cosmetic composition is well-suited for this purpose due to its good applicability, good coloring characteristics and drying behavior. Based on the use of the cosmetic composition as contemplated herein in the hair mascara or the eyebrow coloring agent, the hair, including the eyebrows, can be colored consistently, wherein a film remaining on the hair after the drying of the cosmetic composition is not too dry but not damp and remains shapeable and even combable due to its elasticity.

Therefore, according to a further aspect of the present disclosure, the use of a cosmetic composition as described above for treatment, particularly for temporary coloring of keratinous fibers, is disclosed. In particular, the cosmetic composition can be used for the temporary coloring of the hairline of keratinous fibers.

In addition, according to a further aspect of the present disclosure, a method for treatment of keratinous fibers, particularly for the temporary coloring of keratinous fibers and particularly for the temporary coloring of the hairline of keratinous fibers, is described. The method comprises application of the cosmetic composition described above on keratinous fibers and is carried out, preferably, with a brush, due to its especially consistent application form.

The following example should explain the subject of the present disclosure in greater detail.

### EXAMPLE

A basic recipe for a cosmetic composition as contemplated herein is provided below. All specifications in wt. % are relative to the weight of the cosmetic composition.

Cashmere Red Hair Mascara

| Ingredient | Test sample (wt. %) |
|---|---|
| Cetyl Alcohol | 6.00 |
| Ceteareth-20 | 0.60 |
| Cetearyl Alcohol | 3.40 |
| Ceteareth-20 | 4.00 |
| Cetyl Palmitate | 3.00 |
| PEG-32 (Polyethylene glycol MW 1500) | 1.50 |
| VP/VA Copolymer (60/40) | 0.30 |
| 2-phenoxyethanol | 1.00 |
| 2-ethylhexylglycerol | 0.10 |
| Glycerol | 4.00 |
| Phenyl Trimethicone (Dow Coming 556) | 1.00 |
| Hydrophobized black pigment, including | 1.00 |
| Iron (II, III) oxide (CI 77499) as a pigment | 0.96 |
| core and | |
| triethoxycaprylylsilanes as a hydrophobic | 0.04 |
| coating | |
| Hydrophobized black pigment, including | 7.00 |
| Fe(3) oxide (CI 77491) as a pigment core | 6.72 |
| and | |
| triethoxycaprylylsilanes as a hydrophobic | 0.28 |
| coating | |
| Water | 67.10 |
| | (ad 100.00) |

The cosmetic composition was produced by mixing the fatty alcohols, the cetyl palmitate, the ceteareth-20 and Dow Corning®556 together at approx. 60° C. Then the hydrophobized pigments were added and dispersed homogeneously in the melt retaining an oil phase. Aside from this, polyethylene glycol, glycerol and water were combined and mixed homogeneously. The resulting hydrous phase was also heated to approx. 60° C. Then the oil phase was added to the water phase ender heavy stirring and subsequently homogenized. The mixture obtained was cooled to 40° C. while stirring and then the VP/VA copolymer (60/40) and the preservative (2-phenoxyethanol, 2-ethylhexylglycerol) were added and stirred while cooling down to 25° C.

A black cosmetic composition was obtained, which had a soft, but not self-flowing consistency, with good and consistent application on keratinous fibers (particularly human head hair or eyebrows) by employing, for example, a brush. In particular, the hairline of human head hair could be colored homogeneously without the cosmetic composition drying too quickly. Corrections were possible until the complete drying of the cosmetic composition. After the drying, the cosmetic composition left a dry, flexible and light film on the keratinous fibers. In the process, the individual hairs could be separated and were thus combable while having good formability. Even after the drying, components of the cosmetic composition did not flake off of the keratinous fibers.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic composition for treatment of keratinous fibers comprising:
water,
at least one nonionic oil-in-water emulsifier in a total amount, relative to the weight of the cosmetic composition, of from about 2 to about 10 wt. %,
at least one hydrophobized pigment in a total amount, relative to the weight of the cosmetic composition, of from about 3 to about 15 wt. %,
polyphenylmethylsiloxane and
at least one linear 1-alkanol with from about 14 to about 30 carbon atoms which is solid at 30° C. and a pressure of 1013 mbar,
wherein the amount of soap, relative to the weight of the cosmetic composition, is less than 0.5 wt. %, and
wherein the total amount of polysiloxanes, with the exception of polyphenylmethylsiloxane, relative to the weight of the cosmetic composition, is from about 0 to about 1 wt. %.

2. Cosmetic composition according to claim 1, wherein the amount of polyphenylmethylsiloxane, relative to the weight of the cosmetic composition, is from about 0.05 to about 10 wt. %.

3. Cosmetic composition according to claim 1, wherein the nonionic oil-in-water emulsifier is selected from
ethoxylated C₈-C₂₄ alkanols with from about 10 to about 100 mol of ethylene oxide per mol,
ethoxylated C₈-C₂₄ carboxylic acids with from about 10 to about 100 mol of ethylene oxide per mol,
optionally hydroxylated ethoxylated sorbitan monoesters of linear saturated and unsaturated C₁₂ bis C₃₀ carboxylic acids with from about 20 to about 100 mol of ethylene oxide per mol,
optionally ethoxylated alkyl mono- and oligoglycosides with from about 8 to about 22 carbon atoms in the alkyl radical,
ethoxylated sterines,
partial esters of polyglycerols with from about 2 to about 10 glycerol units and with from about 1 to about 4 saturated or unsaturated, linear or branched optionally hydroxylated C₈- to C₃₀ fatty acid radicals,
and mixtures of the aforementioned substances.

4. Cosmetic composition according to claim 1, further comprising at least one vinyl polymer.

5. Cosmetic composition according to claim 1, further comprising at least one organic solvent selected from glycerol, 1,2-propanediol, 1,3-butanediol and mixtures thereof, wherein the total amount of glycerol, 1,2-propanediol and 1,3-butanediol, relative to the weight of the cosmetic composition is from about 1 to about 15 wt. %.

6. Cosmetic composition according to claim 1 further comprising at least one polyethyleneglycol which is solid at 25° C. and a pressure of 1013 mbar, wherein the total amount of solid polyethylene glycol, relative to the weight of the cosmetic composition, is from about 0.5 to about 10 wt. %.

7. Cosmetic composition according to claim 1 comprising water in a total amount of from about 40 to about 80 wt. %.

8. Cosmetic composition according to claim 1, wherein the linear 1-alkanol with from about 14 to about 30 carbon atoms is selected from myristyl alcohol, cetyl alcohol, stearyl alcohol, arachyl alcohol, behenyl alcohol, erucyl alcohol and brassidyl alcohol, and mixtures thereof.

9. Cosmetic composition according to claim 1 further comprising at least one ester, which is formed from at least of at least one linear or branched, saturated or unsaturated monocarboxylic acid with from about 3 to about 30 carbon atoms, which optionally includes one or multiple hydroxyl groups and at least one linear or branched, saturated or unsaturated alcohol with from about 1 to about 30 carbon atoms and which is solid at 35° C. and a pressure of 1013 mbar.

10. Cosmetic composition according to claim 9, wherein the at least one ester, which is formed from at least of at least one linear or branched, saturated or unsaturated monocarboxylic acid with from about 3 to about 30 carbon atoms, which optionally includes one or multiple hydroxyl groups and at least one linear or branched, saturated or unsaturated alcohol with from about 1 to about 30 carbon atoms and which is solid at 35° C. and a pressure of 1013 mbar, is contained in a total amount of from about 0.1 to about 6 wt. %, relative to the weight of the cosmetic composition.

11. Cosmetic composition according to claim 1, wherein the at least one hydrophobized pigment comprises a pigment core, which is selected from the group of metal oxides, metal oxychlorides, metal hydroxides, and metal oxide hydrates, mixed phase pigments, sulfur-containing silicates, metal sulphides, complex metal cyanides, metal sulphates, metal phosphates, metal chromates, metal molybdates, fluorescent pigments, phosphorescent pigments, metallic effect pigments, phyllosilicates, pearlescent pigments based on natural or synthetic mica, which are coated with one or more metal oxide or a metal oxychloride, and mixtures thereof.

12. Cosmetic composition according to claim 1, wherein the at least one hydrophobized pigment is selected from organic monosilanes.

13. Cosmetic composition according to claim 1, wherein the at least one hydrophobized pigment has a weight ratio of pigment core to hydrophobic coating material in the range of from about 40 to 1 to about 18 to 1.

14. Hair, mascara, or eyebrow coloring agent comprising the cosmetic agent according to claim 1.

15. Method for treatment of keratinous fibers, comprising applying the cosmetic composition according to claim 1 on keratinous fibers.

16. Cosmetic composition according to claim 1, wherein:
the water is present in a total amount, relative to the weight of the cosmetic composition, of from about 60 to 70 wt. %,
the at least one nonionic oil-in-water emulsifier is present in a total amount, relative to the weight of the cosmetic composition, of from about 4.5 to 5.3 wt. %,
wherein the amount of polyphenylmethylsiloxane, relative to the weight of the cosmetic composition, is from about 0.05 to about 10 wt. % 1 to 1.5 wt. %,
the at least one hydrophobized pigment is present in a total amount, relative to the weight of the cosmetic composition, of from about 7 to 9 wt. %,
wherein the amount of soap, relative to the weight of the cosmetic composition, is about 0 wt. %, and
wherein the total amount of polysiloxanes, with the exception of polyphenylmethylsiloxane, relative to the weight of the cosmetic composition, is about 0 wt. %.

17. Cosmetic composition according to claim 1, wherein the nonionic oil-in-water emulsifier is selected from ethoxylated C₈-C₂₄ alkanols with from about 20 to about 30 mol of ethylene oxide per mol.

18. Cosmetic composition according to claim 1, wherein the nonionic oil-in-water emulsifier is selected from ethoxylated cetyl alcohol with from about 20 to about 30 mol of ethylene oxide per mol, ethoxylated stearyl alcohol with from about 20 to about 30 mol of ethylene oxide per mol, and mixtures thereof, and mixtures thereof.

19. Cosmetic composition according to claim 4, wherein the vinyl polymer is at least one vinylpyrrolidone/vinylacetate copolymer, wherein the total amount of vinyl polymer, relative to the weight of the cosmetic composition is from about 0.1 to about 10 wt. %.

20. Cosmetic composition according to claim 1 further comprising:
at least one solid polyethyleneglycol having 25 to 100 ethylene oxide units and which is solid at 25° C. and a pressure of 1013 mbar, wherein the total amount of the solid polyethylene glycol, relative to the weight of the cosmetic composition, is from about 0.5 to about 10 wt. %; and
at least one ester selected from cetyl palmitate, stearyl laurate, cetearyl stearate, myristyl myristate, and mixtures thereof.
